(19) United States
(12) Patent Application Publication
(10) Pub. No.: US 2011/0064787 A1
(43) Pub. Date: Mar. 17, 2011

(54) Detoxifying patch

(76) Inventor: Rick Henry Cabados, Encinitas, CA (US)

(21) Appl. No.: 12/558,883

(22) Filed: Sep. 14, 2009

(51) Int. Cl. A61K 9/70; A61K 33/06; A61P 43/00

(52) U.S. Cl. 424/447; 424/684

(57) ABSTRACT

A detoxifying patch is disclosed that is preferably applied to the soles of the feet as a therapeutic treatment to remove toxins from the body and increase blood circulation. The patch includes a pouch and a backing with an optional foil backing. The pouch contains various ingredients including mandarin wood vinegar, mandarin oil, bamboo vinegar, tourmaline, magnets, magnetic powder and/or zeolite. The patch is secured to a user by an adhesive on the backing or optionally a separate adhesive strip.

### CROSS REFERENCE TO RELATED APPLICATIONS

None.

### STATEMENT REGARDING FEDERALLY SPONSORED RESEARCH OR DEVELOPMENT

This invention was not federally sponsored.

### BACKGROUND OF THE INVENTION

### Field of the invention

This invention relates to the general field of detoxifying patches, and more specifically toward a detoxifying patch that is preferably applied to the soles of the feet as a therapeutic treatment to remove toxins from the body and increase blood circulation. The patch includes a pouch and a backing with an optional foil backing. The pouch contains various ingredients including wood vinegar, bamboo vinegar, tourmaline, saururus chinensis, houttuynia cordata, eucalyptus tree sap, agaricus mushroom, chitosan, cornstarch, magnets, magnetic powder and/or zeolite. The patch is secured to a user by an adhesive on the backing or optionally a separate adhesive strip.

The accumulation of heavy metals, chemicals, and other toxins in the body interfere with its ability to absorb essential minerals and vitamins from foods. Many natural healthcare practitioners believe that these toxins travel down through the body towards the foot. Additionally, circulation of blood and lymphatic fluids reach their furthest point in the soles of the feet before returning back up in the higher portions of the body. Therefore, there is a need for a device and method to remove harmful toxins from the body and improve circulation, preferably from the soles of the feet.

The prior art has several attempts of therapeutic patches placed on various parts of the body to solve this problem. U.S. Pat. No. 6,419,935, to Gueret, teaches a method for cosmetic skin treatment that includes providing a patch configured to be used in both a cleansing mode and a treatment mode. It includes at least one cosmetically active compound and at least one water-absorbent compound. The patch is applied to an area of dry skin so that a polymeric matrix adheres to the skin for a time sufficient to allow at least one impurity in the area of the skin to become attached to the polymeric matrix. When the patch is removed, the at least one impurity is removed with it. However, the method taught by Gueret fails to teach a unique combination of ingredients that includes wood vinegar, bamboo vinegar, tourmaline, saururus chinensis, houttuynia cordata, eucalyptus tree sap, agaricus mushroom, chitosan, cornstarch, magnets, magnetic powder and/or zeolite that efficiently and effectively removes toxins while at the same time increases blood circulation.

U.S. Pat. No. 5,780,047, to Kamiya et al., discloses a patch that comprises a water-soluble adhesive sheet and a water-soluble protective material. It achieves warm-bathing effects on the application site. It improves topical circulatory dynamics and metabolism and, if desired, can apply medicinal substances to affected areas such as stiff necks and shoulders. However, the patch disclosed by Kamiya et al. fails to disclose a unique combination of ingredients that includes wood vinegar, bamboo vinegar, tourmaline, saururus chinensis, houttuynia cordata, eucalyptus tree sap, agaricus mushroom, chitosan, cornstarch, magnets, magnetic powder and/or zeolite that efficiently and effectively removes toxins while at the same time increases blood circulation.

Thus there has existed a long-felt need for a detoxifying patch that can efficiently and effectively remove toxins from the body and increase blood circulation. It should include all natural ingredients that harmonize with the body to remove harmful toxins and not impute any harmful substance into the body. The patch should also increase blood circulation, not only in the immediate area, but throughout the entire body.

The current invention provides just such a solution by having a detoxifying patch that is preferably applied to the soles of the feet as a therapeutic treatment to remove toxins from the body and increase blood circulation. The patch includes a pouch and a backing with an optional foil backing. The pouch contains various ingredients including wood vinegar, bamboo vinegar, tourmaline, saururus chinensis, houttuynia cordata, eucalyptus tree sap, agaricus mushroom, chitosan, cornstarch, magnets, magnetic powder and/or zeolite. The patch is secured to a user by an adhesive on the backing or optionally a separate adhesive strip.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

### SUMMARY OF THE INVENTION

The current invention is a detoxifying patch that includes a pouch with an optional foil backing. The foil backing is used to reflect heat back onto the patched area while minerals, herbs, and other natural substances located within the pouch stimulate the skin to remove toxic waste out of the body and increase blood flow. The materials included inside the pouch can include wood vinegar, bamboo vinegar, tourmaline, germanium, magnets, magnetic powder, saururus chinensis (Chinese lizard's tale), houttuynia cordata (chameleon plant), eucalyptus tree sap, agaricus mushroom, chitosan, cornstarch, and/or zeolite.

It is a principal object of the invention to provide a detoxifying patch that removes toxins from the body.

It is another object of the invention to provide a detoxifying patch that uses all-natural ingredients.

It is an additional object of the invention to provide a detoxifying patch that increases blood circulation.

It is a further object of the invention to provide a detoxifying patch that can be secured to the foot of a user.

It is a final object of this invention to provide a method for removing heavy metals, metabolic waste, microscopic organisms, chemical residue, and other natural and artificial toxins from the body using a detoxifying patch.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

FIG. 1 is a top view of the detoxifying patch.

FIG. 2 is a top view of the detoxifying patch with an optional adhesive strip.

FIG. 3 is a bottom view of the detoxifying patch.

FIG. 4 is a side view of the detoxifying foot patch.

FIG. 5 is a side view of the detoxifying foot patch with an optional adhesive strip.

### DETAILED DESCRIPTION OF THE INVENTION

Many aspects of the invention can be better understood with reference to the drawings below. The components in the drawings are not necessarily drawn to scale. Instead, emphasis is placed upon clearly illustrating the components of the present invention. Moreover, like reference numerals designate corresponding parts through the several views in the drawings.

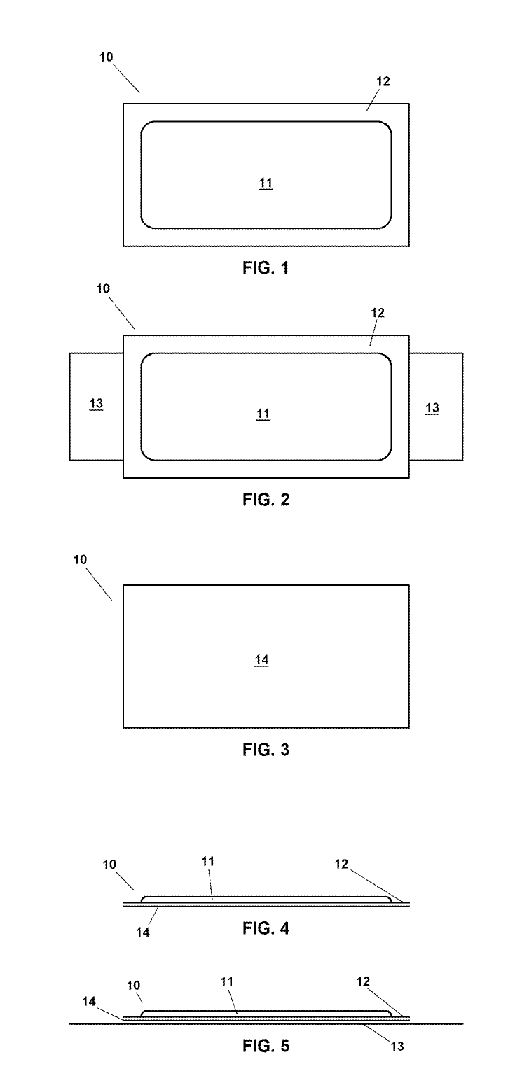

FIG. 1 is a top view of the detoxifying patch. The detoxifying patch 10 includes a pouch 11, or bag, that is connected to a backing 12. The backing 12 can include an adhesive to help secure the detoxifying patch 10 to a user. Other means of attachment to a user are possible, including a separate adhesive patch, as shown in FIG. 2, straps, or the like. However, it is preferable that no adhesives be placed over the pouch 11 as these adhesives may hinder the detoxifying abilities of the patch. The pouch 11 contains the minerals, herbs, and other ingredients that detoxify the body and aid in increasing blood circulation.

FIG. 2 is a top view of the detoxifying patch with an optional adhesive strip. The detoxifying patch 10 includes a pouch 11, or bag, that is connected to a backing 12. In this embodiment of the invention, there is an adhesive strip 13 that is used to secure the detoxifying patch 10 to a user. The pouch 11 contains the minerals, herbs, and other ingredients that detoxify the body and aid in increasing blood circulation.

FIG. 3 is a bottom view of the detoxifying patch. The back of the detoxifying patch 10 includes a foil backing 14. The foil backing 14 is designed to reflect the body's heat back into the area beneath the patch to increase blood circulation and therefore the patches ability to detoxify the body. Various different materials can be used to make the foil backing 14, including copper, tin, and aluminum.

FIG. 4 is a side view of the detoxifying foot patch. The detoxifying patch 10 includes a pouch 11, or bag, that is connected to a backing 12. The backing 12 can include an adhesive to help secure the detoxifying patch 10 to a user. The pouch 11 contains the minerals, herbs, and other ingredients that detoxify the body and aid in increasing blood circulation. The detoxifying patch 10 also includes a foil backing 14 to reflect the body's heat back into the area beneath the patch to increase blood flow and the removal of toxic wastes from the body.

FIG. 5 is a side view of the detoxifying foot patch with an optional adhesive strip. The detoxifying patch 10 includes a pouch 11, or bag, that is connected to a backing 12. In this embodiment of the invention, there is an adhesive strip 13 that is used to secure the detoxifying patch 10 to a user. The pouch 11 contains the minerals, herbs, and other ingredients that detoxify the body and aid in increasing blood circulation. The detoxifying patch 10 also includes a foil backing 14 to reflect the body's heat back into the area beneath the patch to increase blood flow and the removal of toxic wastes from the body.

The pouch 11 can be larger or smaller compared to the backing 12 depending on the specific usage and application of the detoxifying patch 10. Further, the adhesive strip 13 can also be larger or smaller depending on the specific application. The foil backing 14 preferably covers the entire backing 12 to aid in reflecting heat back towards the body, but it can cover less than entire backing 12.

The detoxifying patch is preferably secured to the sole of a user's foot, but can be placed anywhere on the body. The detoxifying patch preferably takes advantage of the biorhythmic network by removing toxins from the body though the more than 60 acupuncture points found on the soles of the feet. Many alternative health practitioners provide alternative healing methodologies that rely on the belief that toxins may be safely removed through acupuncture points in the feet. Toxic waste is passed out of the body through expanded pores stimulated by the detoxifying patch. In a process similar to osmosis, the toxins diffuse out of the body and are absorbed by the patch.

The pouch is filled with a unique blend of ingredients to create an efficient and effective detoxifying patch that effectively removes toxins from the body as well as increases blood circulation. These ingredients can include, but are not limited to, bamboo vinegar, wood vinegar, tourmaline, zeolite, saururus chinensis, houttuynia cordata, eucalyptus tree sap, agaricus mushroom, chitosan, germanium, magnets, magnetic powder, and cornstarch. The pouch can also include other herbs and/or magnetic powder evenly distributed throughout the pouch. Some or all of the ingredients can also be suspending in a foam layer to keep them evenly distributed.

Bamboo vinegar has unique herbal properties that include the ability to assist in the discharge of toxic waste from the body through the skin. The bamboo sap, which the bamboo vinegar is derived from, is rich in minerals and trace elements that assist in the detoxification.

Wood vinegar, or pyroligneous acid, derived from the fluid or sap of tree cells, includes permeability and sterilizing properties. It has been confirmed that the drawing power of wood vinegar can be used for physiotherapy, including minor pain relief and decreasing swelling in the body.

Tourmaline is a mineral that emits negative ions and far-infrared rays. These properties have been known to help improve circulation, relieve stress, increase mental alertness, and strengthen the immune system. The energy produced by this mineral has been shown to increase the metabolism of the body on a molecular level thereby allowing the cell walls to expand and the easier transfer of waste toxins across these membranes into the surrounding blood and lymph systems.

Zeolite is a mineral with a micro-porous structure that has been shown to remove toxins, free radicals, and metals from the body. It is one of only two minerals that is negatively charged by nature. This negative charge attracts toxins like a magnet. Further, the cage like structure of the zeolite traps these body harming toxins and wastes after they are extruded from the body.

Saururus Chinensis, or Chinese lizard's tail, is a native East India herb that has been shown to remove toxic heat and encourage diuresis. Houttuynia cordata, or chameleon plant, is a perennial herb from the mountainous regions of eastern Asia. This herb is regarded for its antibacterial properties and its ability for draining infections.

Eucalyptus tree sap is known as one of the most powerful antiseptics in its class. These effects are magnified when the eucalyptus tree sap is aged, as ozone is formed when exposed to air. The eucalyptus tree sap destroys unhealthy flora and microbes that it comes in contact with.

Agaricus blazei murill, or agaricus mushroom, originated in the rain forests of Brazil. Several studies have documented this mushroom's ability to activate and stimulate various components of the immune system.

Chitosan is derived from chitin, the main fiber component of crustacean shells. It has unique absorption capabilities that aid in the isolating fatty tissues and assisting in dispelling toxins and wastes out of the body.

Cornstarch is used as a complementary ingredient because of its excellent absorption capabilities. It is an important ingredient in helping to bind extracted toxins to the patch thereby preventing re-absorption back through the skin of the body.

Other ingredients can include germanium, magnets, and magnetic powders. Magnets and magnetic powder can be included to create a magnetic field that penetrates the skin around the patch. This can have an additional affect on the blood flow to increase circulation throughout the body.

Some or all of these ingredients are combined to create a unique patch that is effective and efficient at removing toxins from the body and increasing blood circulation. The combination of these ingredients, especially tourmaline and zeolite, into a patch for easy removal of toxins and improved circulation create a unique and beneficial detoxifying patch. In fact, the addition of naturally negatively charged tourmaline and zeolite into a patch creates an especially efficient and effective means to remove lead, arsenic, and other heavy metals.

A preferred combination of ingredients for the detoxifying patch includes 17% bamboo vinegar, 4% zeolite, 10% cypress vinegar, 10% tourmaline, 5% agaricus mushroom, 9% achyranthis radix, 9% paeoniae rubrae radix, 9% Siberian ginseng, 9% Artemisia iwayomogi, 9% caragana sinica, and 9% platicodon grandiflorum.

A user takes the patch and applies it to a part of the body. Preferably, the detoxifying patch is secured to the bottom of the foot where most of the nerves in the body end. Preferably, the patch is worn for approximately 12 hours, and then discarded. Beneficial effects can occur within as little as 2 hours and continue on for as much as 24 hours, but the optimal range is between 8 and 16 hours. The detoxifying patch can be worn anytime, such as while walking, running, or during other physical activity or preferably it can be worn while sleeping. As the detoxifying patch removes toxins, it will become discolored and turn from a white to a dark brown or gray. However, after several uses of the detoxifying patch, the amount of discoloration should decrease, as fewer toxins are present in, and therefore removed from, the body.

What I claim is:

1. A detoxifying patch comprising:
a backing, a pouch, and ingredients, where the ingredients of the detoxifying patch are located within the pouch,
where the ingredients comprise tourmaline and zeolite.

2. The detoxifying patch of claim 1, further comprising a foil backing.

3. The detoxifying patch of claim 1, where at least a portion of the backing includes an adhesive.

4. The detoxifying patch of claim 1, further comprising an adhesive strip.

5. The detoxifying patch of claim 1, where the ingredients further comprise bamboo vinegar and wood vinegar.

6. The detoxifying patch of claim 5, where the ingredients further comprise eucalyptus tree sap.

7. The detoxifying patch of claim 1, where the ingredients further comprise saururus chinensis and houttuynia cordata.

8. The detoxifying patch of claim 1, where the ingredients further comprise agaricus mushroom.

9. The detoxifying patch of claim 1, where the ingredients further comprise chitosan.

10. The detoxifying patch of claim 1, where the ingredients further comprise cornstarch.

11. A detoxifying patch comprising:
a backing, a pouch, and ingredients, where the ingredients of the detoxifying patch are located within the pouch,
where the ingredients comprise tourmaline, zeolite, saururus chinensis, houttuynia cordata, agaricus mushroom, and chitosan.

12. The detoxifying patch of claim 11, where the ingredients further comprise bamboo vinegar, wood vinegar, and eucalyptus tree sap.

13. The detoxifying patch of claim 11, where the ingredients further comprise cornstarch.

14. The detoxifying patch of claim 11, further comprising a foil backing.

15. The detoxifying patch of claim 11, where at least a portion of the backing includes an adhesive.

16. The detoxifying patch of claim 11, further comprising an adhesive strip.

17. A method for removing toxins from a body comprising the following steps:
a) obtaining a detoxifying patch, where the detoxifying patch comprises a backing, a pouch, and ingredients, where the ingredients of the detoxifying patch are located within the pouch, where the ingredients comprise tourmaline and zeolite,
b) securing the detoxifying patch to a part of the body,
c) leaving the detoxifying patch in place for at least 2 hours and less than 24 hours,
d) removing the patch from the body.

18. The method of claim 17, where the detoxifying patch is secured to the sole of a foot of the body.

19. The method of claim 17, where in step c) the detoxifying patch is left in place for at least 8 hours and less than 16 hours.

20. The method of claim 17, where the ingredients of the patch further comprise bamboo vinegar, wood vinegar, saururus chinensis, houttuynia cordata, eucalyptus tree sap, agaricus mushroom, chitosan, and cornstarch.
